(19) United States
(12) Patent Application Publication
(10) Pub. No.: US 2012/0179035 A1
(43) Pub. Date: Jul. 12, 2012

(54) MEDICAL DEVICE WITH MOTION SENSING

(75) Inventor: Aurelie Boudier, Buc (FR)

(73) Assignee: GENERAL ELECTRIC COMPANY, Schenectady, NY (US)

(21) Appl. No.: 12/986,901

(22) Filed: Jan. 7, 2011

(51) Int. Cl. A61B 8/00; H01J 31/49; A61N 7/00

(52) U.S. Cl. 600/439; 378/189

(57) ABSTRACT

In one embodiment, a medical system includes an emitter or a detector configured to interface with a patient and integrated with a housing of a handheld medical instrument, a motion detection device integrated with the housing and configured to detect motion of the handheld medical instrument and convert the motion to motion data, and a processor configured to receive the motion data and programmed to issue a command related to control of the medical system based on a correlation between the motion data and the command.

### BACKGROUND OF THE INVENTION

The present invention generally relates to medical systems and, more particularly, to systems and methods involving medical devices with motion sensing features.

Numerous different diagnostic and therapeutic medical techniques (e.g., ultrasound and X-ray procedures) utilize handheld medical instruments, such as electronic probes (e.g., portable exploratory devices), hand held therapeutic devices, or the like. Certain handheld medical instruments may operate independently or in coordination with a main unit that controls or monitors aspects of the handheld device. Such devices may be capable of performing different functions based on user selection of a desired functionality. For example, ultrasound systems, which generally utilize cyclic sound pressure for diagnostic (e.g., diagnostic imaging) and treatment (e.g., tissue manipulation) purposes, typically include an ultrasound probe or emission device and a separate main unit (e.g., a control and/or monitoring unit) that cooperate to provide different functions. The main unit typically includes a processor and control programming. Ultrasound systems may include a probe with different transducers that may be activated to allow for performing various different types of ultrasound emission. Specifically, for example, certain settings on the main unit of a traditional ultrasound system may be manipulated to cause the related ultrasound probe to emit a ultrasound wavelengths in a selected range or to otherwise control or monitor the ultrasound probe.

Under certain circumstances, it can be difficult for a physician or other healthcare worker to adjust the settings of a traditional medical system to enable different functionalities or otherwise operate the system. Indeed, a second healthcare worker or an assistant is traditionally required in such situations. For example, a healthcare worker may be utilizing a probe in one hand and a different medical instrument in the other hand, which may make it difficult to activate system controls (e.g., system switches and buttons) in order to change settings or operate the medical system. Similarly, in certain environments, access to control features may be difficult due to sterilization requirements or a crowded work space. For example, in certain situations, the main unit of an ultrasound system may not be capable of sterilization and, thus, must be kept out of the room or area in which the related ultrasound probe is being utilized. Accordingly, an assistant may be required to activate or deactivate control features of the ultrasound system to change functional characteristics of the system.

### BRIEF DESCRIPTION OF THE INVENTION

In accordance with one embodiment, a medical system includes an emitter or a detector configured to interface with a patient and integrated with a housing of a handheld medical instrument. The medical system also includes a motion detection device integrated with the housing and configured to detect motion of the handheld medical instrument and convert the motion to motion data. Further, the medical system includes a processor configured to receive the motion data and programmed to issue a command related to control of the medical system based on a correlation between the motion data and the command.

In accordance with another embodiment, a method of operating a medical system includes emitting or detecting waves from a handheld medical instrument to facilitate a therapeutic or diagnostic procedure. The method also includes detecting movement of the handheld medical instrument with a motion detection device and providing motion data based on the movement. Further, the method includes communicating the motion data to a processor, and receiving a command related to control of the handheld medical instrument in response to the motion data.

In accordance with a further embodiment, an ultrasound system includes a handheld ultrasound device and a main unit. The handheld ultrasound device includes a housing, an emitter integral with the housing and configured to emit ultrasound into a target, a motion detection device integral with the housing, and a first communication feature configured to transmit motion data from the motion detection device. The main unit includes a second communication feature configured to receive the motion data from the first communication feature, and a memory and a processor configured to perform various operations based on detection of a motion or series of motions of the handheld medical instrument.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical overview of a medical system in accordance with one embodiment;

FIG. 2 illustrates an ultrasound probe being employed by a user to initiate commands to control the probe or other aspects of an associated ultrasound system by sweeping the probe in different directions in accordance with one embodiment;

FIG. 3 illustrates an ultrasound probe being employed by a user to initiate commands to control the probe or other aspects of an associated ultrasound system by aiming the probe in accordance with one embodiment;

FIG. 4 illustrates an ultrasound probe being employed by a user to initiate commands to control the probe or other aspects of an associated ultrasound system by moving the probe in a circle in accordance with one embodiment;

FIG. 5 illustrates a medical system and a sterile environment with a probe of the system being present in the sterile environment and the related main unit located outside of the sterile environment, wherein the probe is being employed by a user to issue control commands in accordance with one embodiment;

FIG. 6 illustrates a medical system and a sterile environment with a probe of the system being present in the sterile environment and the related main unit loaced outside of the sterile environment, wherein the probe is being employed by a user to issue control commands in accordance with one embodiment; and

FIG. 7 illustrates a method in accordance with one embodiment.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Moreover, while the term “exemplary” may be used herein in connection to certain examples of aspects or embodiments of the presently disclosed technique, it will be appreciated that these examples are illustrative in nature and that the term “exemplary” is not used herein to denote any preference or requirement with respect to a disclosed aspect or embodiment. Further, any use of the terms “top,” “bottom,” “above,” “below,” other positional terms, and variations of these terms is made for convenience, but does not require any particular orientation of the described components.

Embodiments of the present disclosure are directed to a portable medical instrument, such as a handheld probe or therapeutic device that includes or cooperates with a motion detection feature to enable operation or configuration of the portable medical instrument. Embodiments include features that function to perform therapeutic or diagnostic actions (e.g., X-ray imaging, ultrasound imaging, or tissue manipulation), and features that enable detection of movement of the medical instrument and provide corresponding motion data. Further, embodiments include a processor that is configured to control, configure, or adjust operational settings of the medical instrument based on the detected movement. For example, certain gestures (e.g., a movement or series of movements) may be detected and an associated operation (e.g., emitting ultrasound at a different wavelength) may be initiated. Additionally, embodiments include actuation features (e.g., buttons or switches) on the handheld medical instrument that may be utilized along with the motion data to provide control commands.

It should be noted that the term medical instrument is used herein to refer to a medical device that is configured to interact with a target, such as a patient's tissue. For example, a medical instrument may include an ultrasonic probe that emits ultrasonic waves into a patient's tissue through an interface and detects the waves after passage through the tissue to facilitate imaging. In other embodiments, the medical instrument may include a therapeutic ultrasonic device that is handheld and capable of emitting different ultrasonic wavelengths into a patient's tissue for therapeutic purposes. Further, the medical instrument may include features that facilitate use with various different medical technologies, such as ultrasound, X-ray, computed tomography, tomonsynthesis, magnetic resonance imaging, positron emission tomography, combinations of such technologies, and so forth. For example, present embodiments may be utilized in conjunction with a handheld X-ray device such as that disclosed in U.S. application Ser. No. 12/786,323, which is hereby incorporated by reference in its entirety.

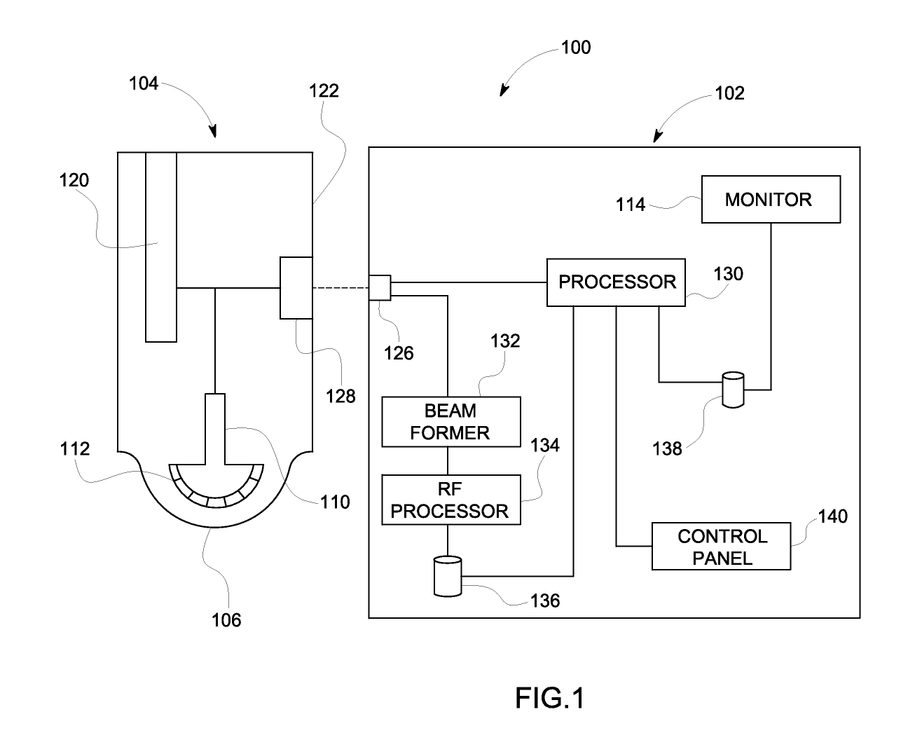

With the foregoing comments in mind and turning to FIG. 1, this figure illustrates diagrammatically an ultrasound system 100 that may be used to acquire and process ultrasonic images. The ultrasound system 100 is an example of a medical system that may be utilized in accordance with present embodiments. In other embodiments, various different types of medical systems may be utilized (e.g., X-ray systems). The ultrasound system 100 includes a main unit 102 and a probe 104. The probe 104 includes an interface 106 (e.g., a scan head) that includes an emitter and/or a detector. In some embodiments, the probe 104 may include only an emitter. In other embodiments, a single feature performs the function of both the emitter and the detector. For example, in the illustrated embodiment, one or more transducers 110 serve as both the emitter and the detector. The transducers 110 are configured to emit ultrasonic signals into a target (e.g., a body or volume). When the ultrasonic signals are back-scattered from density interfaces and/or structures in the target (e.g., blood cells and muscular tissue), echoes are produced that return to and get detected by the transducer 110. In one embodiment, the transducer 110 includes one or more arrays of elements 112 (e.g., piezoelectric crystals) within or formed as part of the transducer 110. It should be noted that a variety of transducers 110 and geometries may be utilized in accordance with present embodiments. Further, in other embodiments, various different types of emitters and/or detectors may be utilized to perform diagnostic and therapeutic operations.

The ultrasound system 100 may be capable of various different modes of operation. For example, the probe 104 may be capable of emitting various different wavelengths of ultrasound or different pulse sequences. Further, the ultrasound system 100 may include control features that enable deactivation of the probe 104 or initiation of a different display on a monitor 114 of the main unit 102. Further, as indicated above, the ultrasound system 100 is configured to facilitate detection of movement of the ultrasound probe 104. Certain movements or gestures are associated with different commands, which enables the system 100 to be adjusted or configured based on movement of the probe 104. In the illustrated embodiment, this is achieved, in part, by a motion detection feature 120 that is integral with the ultrasound probe 104. For example, the motion detection feature 120 may be disposed within a housing 122 of the probe 104, coupled with the housing 122 of probe 104, or otherwise integrated with the probe 104. The motion detection feature 120 may include any of various features that facilitate motion detection. In one embodiment, the motion detection feature 120 includes an accelerometer, a light emitter (e.g., an infrared light emitter) that functions with a separate detector, or a combination of such features. In some embodiments, the motion detection feature may be separate from the probe 104 and may be a component of or interact directly with the main unit 102. For example, the motion detection feature 120 may include a device that includes a camera configured to track the motion of the probe 104. Further, in some embodiments, a combination of features may be utilized to track movement of the probe 104.

In the illustrated embodiment, the main unit 102 and the probe 104 each include a communication feature 126, 128. These communication features 126, 128 cooperate to facilitate transmission of data between the main unit 102 and the probe 104. This may be achieved via a communication cable or wirelessly (e.g., via a wireless local area network). In one embodiment, the communication features 126, 128 include communication ports that can be communicatively coupled via a transmission cable. In another embodiment, the communication features 126, 128 include transmission devices that can be communicatively coupled with a wireless network.

The communication features 126, 128 may facilitate activation, operation, and adjustment of settings for the probe 104 and/or the main unit 102. For example, the communication features 126, 128 may enable communication of instructions to the probe 104 from the main unit 102, and communication of data or signals from the probe 104 to the main unit 102 for processing. In one embodiment, the communication feature 126 of the main unit 102 transmits signals to the probe 104 to activate certain features of the probe 104 that are integral with (e.g., contained within, extending from, or otherwise integrated with) the probe 104. For example, the main unit 102 may transmit drive signals to the probe, wherein the drive signals drive one or more arrays of the elements 112 (e.g., piezoelectric crystals) within or formed as part of the one or more transducers 110 of the probe 104 to emit pulsed ultrasonic signals into a target (e.g., a body or volume). The drive signals may be controlled by a processor 130 of the main unit 102. In other embodiments, the processor 130 may be disposed within the probe 104 and the probe 104 may be self-driven.

Signals related to the echoes of the pulsed ultrasonic signals detected by the probe 104 may be transmitted, via the communication features 126, 128, from the probe 104 to the main unit 102 for processing. Specifically, the signals related to the echoes may be transmitted from the communication feature 126 to a beamformer 132, which performs beamforming on the signals and outputs a radio frequency (RF) signal to an RF processor 134. The RF processor 134, which processes the RF signal, may include a complex demodulator that demodulates the RF signal to form in-phase quadrature (IQ) data pairs representative of the echo signals. The RF or IQ signal data may then be routed directly to an RF/IQ buffer 136 for storage (e.g., temporary storage). The processor 130 is configured to processes the acquired ultrasound information (e.g., RF signal data or IQ data pairs) and prepares frames of ultrasound information for presentation on the monitor 114. An image buffer 138 may be included for storing processed frames of ultrasound information. Acquired ultrasound information may be processed in substantially real-time during a scanning session or stored in the RF/IQ buffer 136 and processed by the processor 130 later. The processor 130 is capable of performing one or more processing operations on the acquired ultrasound information according to a plurality of selectable ultrasound modalities. As discussed below, a user may be able to control operation of the ultrasound system 100, including ultrasound modalities and functionality of the probe 104, via movement of the probe 104, which is detected by the motion detection feature 120. Also, control may be achieved via user input directly on a control panel 140 of the main unit 102.

Data provided by the motion detection feature 120 may be transmitted between the probe 104 and the main unit 102 with the communication features 126, 128 and used to control operation of the ultrasound system 100. In accordance with present embodiments, the probe 104 can communicate with the main unit 102 and command the device by the perception of movements of the probe 104. For example, data related to detected motion of the probe 104 may be transmitted from the motion detection feature 120 to the processor 130 of the main unit 102 (or a processor of the probe 104 in a self-contained embodiment) to indicate that a mode of operation should be changed (e.g., a different wavelength of ultrasound should be emitted or the system should be turned off).

The motion detection feature 120 may include one or a combination of various different features that work to detect movement of the probe 104. In one embodiment, the motion detection feature 120 includes one or more accelerometers. In some embodiments, the motion detection feature 120 includes an infrared light emitter and an associated detection, and information related to light emitted by the infrared light source and detected by a component (e.g., an infrared light detector) of the main unit 102 may be transmitted from the main unit 102 to the probe 104. Further, in some embodiments, the main unit 104 may include or interface with a motion tracking feature, such as a camera configured to track movement of the probe, and data produced by such a feature may be utilized to produce command signals that are transmitted to the probe via the communication features 126, 128. Assembled information from the one or more motion detection features 120 may be utilized to identify a relative position or series of positions of the probe 104, and this information may correlate to programmed instructions for operation of the ultrasound system 100.

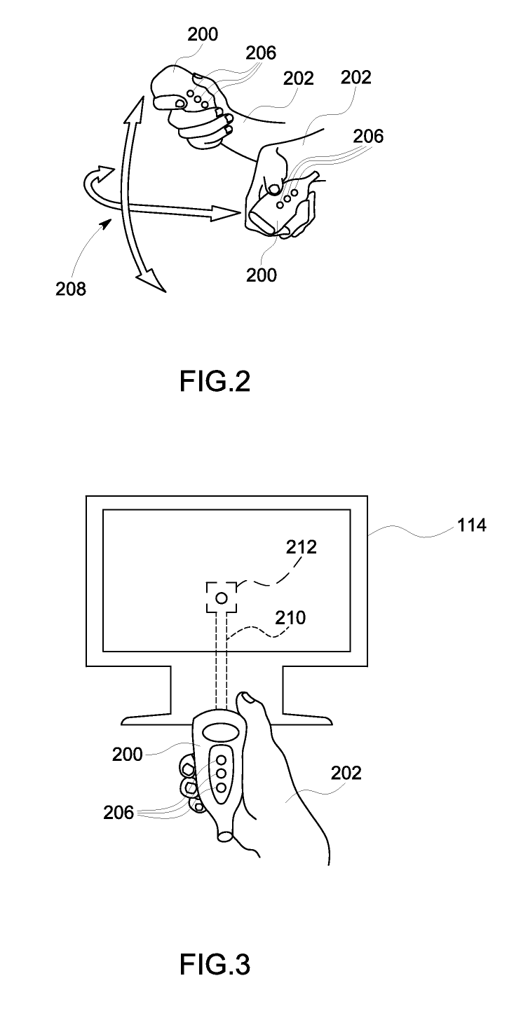

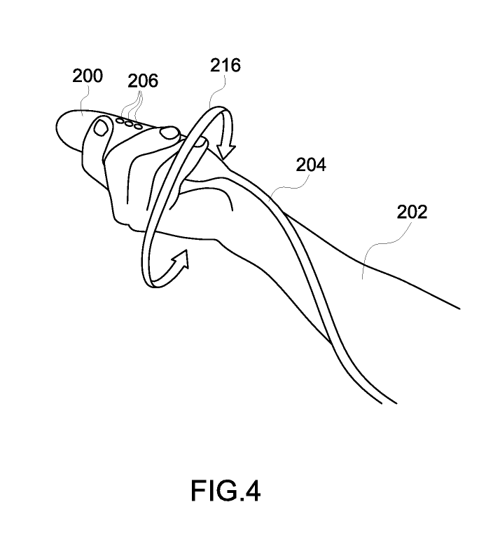

FIGS. 2-4 illustrate an ultrasound probe 200 being employed by a user 202 to initiate commands to control the probe 200 or other aspects of an associated ultrasound system in accordance with present embodiments. The probe 200, as illustrated in FIGS. 2 and 3 is configured for wireless operation, while the probe illustrated in FIG. 4 includes a cable 204. More specifically, the probe 200, as illustrated in FIGS. 2 and 3 are configured to communicate with a main unit wirelessly or are self-contained, and the probe 200 illustrated in FIG. 4 communicates with a main unit via the cable 204. It should be noted that, in the illustrated embodiments, the probe 200 includes a plurality of buttons 206. The buttons 206 are representative of actuation features or devices (e.g., switches, buttons, and scroll wheels) that may be employed as input features in accordance with present embodiments. In addition to the motion of the probe 200, these buttons 206 or other actuation features may be utilized to provide commands alone or in combination with probe movement. For example, each button 206 may initiate a command and each button 206 may modify a command indicated by a particular motion pattern of the probe 200.

FIG. 2 illustrates one type of probe motion that may be detected and utilized to initiate a command in accordance with present embodiments. Specifically, the user 202 is maneuvering the probe 200 in sweeping patterns to essentially form a cross-like pattern 208. This series of motions may be detected by the motion detection feature or features 120 and the processor 130 may interpret the series of motions as corresponding to a delete command, a command to power down the system, a command to adjust performance, or any of various different control commands. If one or more of the buttons 206 are pressed during the motion associated with this pattern 208, a different or slightly modified command may be initiated. For example, pressing a button in addition to the motion of the pattern 208 may cause the related command to initiate more quickly. Accordingly, the user 202 can employ the probe to provide commands without accessing an associated main unit. For example, when the area in which the procedure is being performed blocks ready access to the main unit but enables access to the probe.

FIG. 3 illustrates the probe being aimed at the monitor 114 of the ultrasound system 100. In this embodiment, the motion detection feature 120 includes a light emitter that is emitting an infrared beam 210 and the monitor 114 includes infrared detection features that may cooperate with other motion detection features to determine where the user 202 is pointing the probe 200. Indeed, as instructed by the processor 130, the monitor 114 may provide indications 212 (e.g., highlighting of selected icons) of where the probe 200 is perceived to be pointing. This enables the user 202 to remotely select from a display menu on the monitor 114. Accordingly, the user 202 can employ the probe 200 from a location (e.g., a sterile environment) that does not readily enable access to controls on the main unit but that allows the presence of the probe.

FIG. 4 illustrates the user 202 moving the probe 200 in a generally circular pattern 216, which may be detected and interpreted as one of various different control commands. The frequency of the movement may be utilized as part of the command as well. For example, a circular motion may correspond to a command to scroll through a menu. When the circular motion is in a first direction, the command may indicate scrolling down. When the circular motion is in a second direction that is opposite the first direction, the command may indicate scrolling up. Further, the rapidity of the motion may correspond to a command to scroll faster. As will be understood, various different commands may be associated with various different motions. Further, circular motions in conjunction with actuation of one or more of the buttons 206 may provide modified commands.

It is now recognized that physicians and other healthcare workers often perform medical procedures in situations that can create difficulties related to control or monitoring of certain medical instruments utilized during such procedures. Indeed, the circumstances surrounding certain procedures that include utilization of a probe and a main unit may make it difficult for a healthcare provider to manipulate certain aspects of the main unit or probe to perform different tasks. For example, ultrasound procedures may be performed as an integral part of procedures that can be complex and crowded (e.g., rheumatology and anesthesia procedures), and it may be difficult to access or otherwise control the main unit while utilizing the probe. Indeed, in addition to manipulating the probe with one hand, the healthcare worker may also be performing a medical procedure (e.g., insertion of a catheter) which may occupy the healthcare worker's other hand. In certain situations, it can be difficult for a healthcare worker using traditional equipment to face the device's display, be close to the patient, and have access to controls on the device while holding the probe. For example, control features on a main unit may be on an opposite side of a patient's bed from the position of a healthcare worker, which makes the control features difficult to access.

Further, in some procedures, an ultrasound probe must be utilized in a sterile environment (e.g., chirurgical rooms). In such situations it becomes difficult for the healthcare worker to manage the ultrasound device, which cannot be present in the room or is otherwise difficult to access due to the inability to sterilize system components. While the probe may be sterilized (e.g., covered with a sterile sock) and placed within the sterile field, the physician cannot touch the controls on the main unit because of potential contamination and because the system is generally outside of the room or difficult to access. Thus, an assistant is typically utilized in such situations. As discussed in the examples below, present embodiments address situations wherein it is difficult for a healthcare worker to access certain control feature while utilizing an ultrasound probe by allowing the healthcare worker handling the probe to move the probe in certain patterns to control operation of the probe and/or main unit.

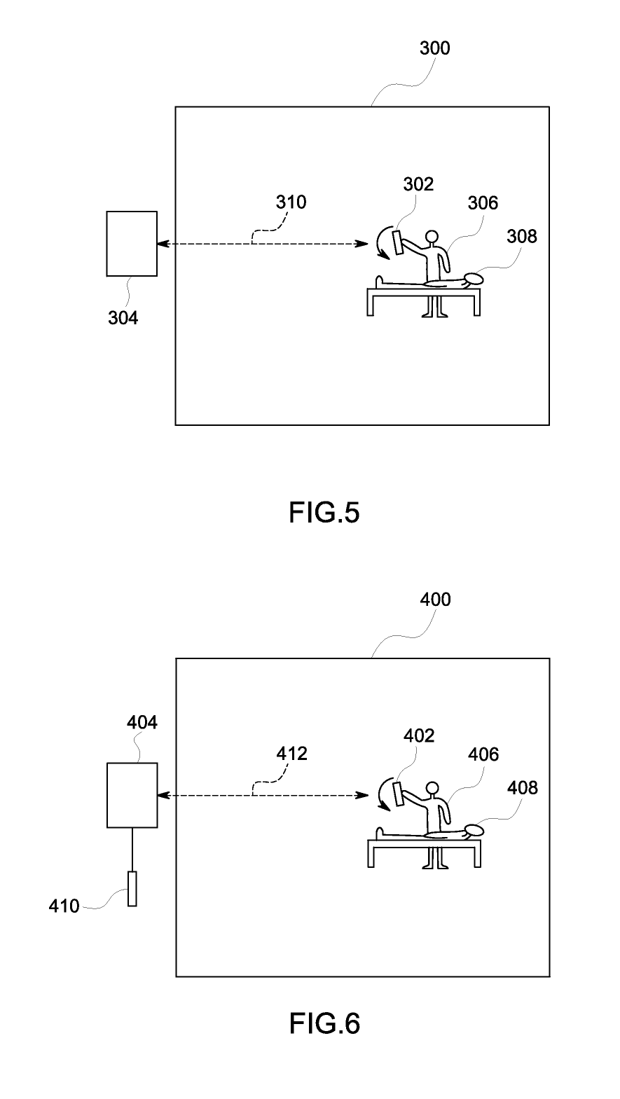

FIG. 5 illustrates a sterile environment 300 wherein a probe 302 is present in the sterile environment 300 and the related main unit 304 is located outside of the sterile environment 300. It should be noted that this situation is representative of various scenarios in which the main unit 304 is substantially inaccessible during a procedure but the probe 302 is readily available. Indeed, in some embodiments, the main unit 304 may be inaccessible due to circumstances that are related or unrelated to sterilization. For example, the main unit 304 may simply be positioned away from the area in which the procedure is performed (e.g., positioned on the other side of the patient's bed from the healthcare worker). As illustrated in FIG. 5, a user 306 (e.g., a healthcare worker) is performing a procedure on a patient 308 using the probe 302. While the main unit 304 is inaccessible in the illustrated embodiment, in other embodiments, the healthcare worker 306 may simply find it difficult to reach the controls of the main unit 304 while performing the procedure due to a crowded room or engagement of one hand with the probe 302 and the other hand with another aspect of the procedure. Accordingly, the probe 302 illustrated in FIG. 5 includes a motion detection feature and communication features that coordinate to identify motion of the probe 302 and transmit signals indicative of the motion to the main unit 304, as indicated by signal transmission 310, which may be over a cable or wireless. Once received, the signals indicating motion of the probe 302 may be interpreted by the main unit 304 as corresponding to particular control commands. For example, a particular motion or gesture may indicate that the user 306 wants to change the mode of operation of the probe 302 such that it emits a different level of ultrasound. In this situation, the main unit 304 transmits a command to the probe to change operating modes. Similarly, various different aspects of the main unit 304 and the probe 302 may be controlled by movement of the probe 302.

FIG. 6 depicts an environment and features similar to the environment and features depicted in FIG. 5. Indeed, FIG. 6 illustrates a sterile environment 400 wherein a probe 402 is present in the sterile environment 400 and the related main unit 404 is located outside of the sterile environment 400. As with the embodiment illustrated in FIG. 5, it should be noted that this situation is representative of various scenarios in which the main unit 404 is substantially inaccessible during a procedure but the probe 402 is readily available. As illustrated in FIG. 6, a user 406 (e.g., a healthcare worker) is performing a procedure on a patient 408 using the probe 402. While the main unit 404 is inaccessible in the illustrated embodiment, in other embodiments, the healthcare worker may simply find it difficult to reach the controls of the main unit 404 while performing the procedure (e.g., due to the user's operation of multiple devices). To address this, the embodiment illustrated in FIG. 6 includes a motion detector 410 and communication features. These features coordinate to identify motion of the probe 402 and transmit related signals, as indicated by signal transmission 412, which may be over a cable or wireless. The detected motion of the probe 402 may be interpreted by the main unit 404 as corresponding to particular control commands. For example, a particular motion or gesture may indicate that the user 406 wants to change the mode of operation of the probe 402 such that it emits a different level of ultrasound. In this situation, the main unit 404 transmits a command to the probe 402 to change operating modes. Similarly, as with the embodiment described with respect to FIG. 5, various different aspects of the main unit 404 and the probe 402 may be controlled by movement of the probe 402.

Unlike the embodiment illustrated in FIG. 5, the motion detector 410 illustrated in FIG. 6 is at least partially separate from the probe 402 and functions to monitor movements of the probe 402. For example, the motion detector 410 may include a camera and a processor with motion tracking programming that enable tracking of movement of the probe 402. In one embodiment, the probe 402 may include certain features that are tracked by a camera feature or detector of the motion detector 410. For example, the probe 402 may emit infrared light that is tracked by the motion detector 410. In some embodiments, a combination of features such as those described in FIG. 5 and FIG. 6 may be utilized to track the probe 402. For example, the probe 402 may include an accelerometer that is local to the probe 402 and an infrared emitter that is tracked by a detector that is separate from the probe 402 (e.g., integral with the main unit 404).

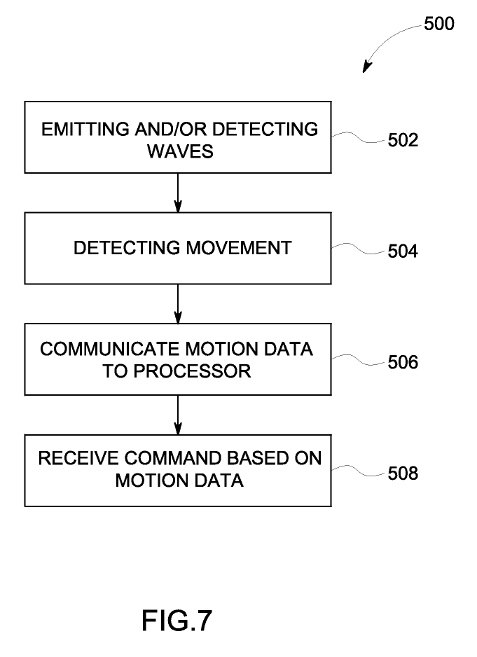

FIG. 7 illustrates a method 500 in accordance with one embodiment. The method includes emitting or detecting waves from a handheld medical instrument to facilitate a therapeutic or diagnostic procedure, as illustrated by block 502. This may include emitting and/or detecting X-rays, ultrasound, and the like. As represented by block 504, the method 500 also includes detecting movement of the handheld medical instrument with a motion detection device and providing motion data based on the movement. This may be achieved with any of various different motion detection features, such as an accelerometer. Further, as illustrated by block 506, the method 500 includes communicating the motion data to a processor. This may include transmission via a cable or wireless transmission of the motion data to a processor that is part of a main unit or integral with the handheld medical instrument. Additionally, as represented by block 508, the method 500 includes receiving a command related to control of the handheld medical instrument in response to the motion data.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A medical system, comprising:
an emitter or a detector configured to interface with a patient and integrated with a housing of a handheld medical instrument;
a motion detection device integrated with the housing and configured to detect motion of the handheld medical instrument and convert the motion to motion data; and
a processor configured to receive the motion data and programmed to issue a command related to control of the medical system based on a correlation between the motion data and the command.

2. The medical system of claim 1, comprising the detector, wherein the detector comprises an X-ray detector.

3. The medical system of claim 1, wherein the processor is disposed within a main unit separate from the handheld medical instrument.

4. The medical system of claim 3, comprising communication features configured to communicatively couple the handheld medical instrument with the main unit via a cable or wirelessly.

5. The medical system of claim 1, wherein the processor is disposed within the housing.

6. The medical system of claim 1, comprising the emitter and the detector, wherein the emitter is configured to emit ultrasound into a target and the detector is configured to detect the ultrasound after passing through the target.

7. The medical system of claim 1, comprising at least one actuation feature on the handheld medical instrument configured to provide a status of the actuation feature to the processor via a communication feature.

8. The medical system of claim 1, comprising an interface configured to facilitate emission of waves into a patient from the emitter or detection of waves from the patient with the detector.

9. The medical system of claim 1, comprising a light emitter assembled with the housing and configured to transmit a light beam indicative of a direction in which the handheld medical instrument is pointed to a light detection feature separate from the handheld medical instrument.

10. The medical system of claim 1, wherein the processor comprises a control feature configured to change a mode of operation of the handheld medical instrument based detection of a motion or series of motions indicated by the motion data and based on a status of actuation features of the handheld medical instrument.

11. The medical system of claim 1, wherein the motion detection device comprises an accelerometer.

12. A method, comprising:
emitting or detecting waves from a handheld medical instrument to facilitate a therapeutic or diagnostic procedure;
detecting movement of the handheld medical instrument with a motion detection device and providing motion data based on the movement;
communicating the motion data to a processor; and
receiving a command related to control of the handheld medical instrument in response to the motion data.

13. The method of claim 12, wherein detecting the movement of the handheld medical instrument with the motion detection device comprises detecting the movement with at least one accelerometer.

14. The method of claim 12, comprising processing the motion data by comparing the motion data to a table of motion data with correlations to control commands with a programmed processor.

15. The method of claim 12, comprising communicating the motion data and data related to a status of at least one actuation feature of the handheld medical instrument from the handheld medical instrument to a main unit via a cable or wirelessly.

16. The method of claim 12, comprising communicating the motion data from the handheld medical instrument to a main unit that is outside of a sterile area while the handheld medical instrument is within the sterile area.

17. An ultrasound system, comprising:
a handheld ultrasound device, comprising:
a housing;
an emitter integral with the housing and configured to emit ultrasound into a target;
a motion detection device integral with the housing; and
a first communication feature configured to transmit motion data from the motion detection device; and
a main unit, comprising:
a second communication feature configured to receive the motion data from the first communication feature; and
a memory and a processor configured to perform various operations based on detection of a motion or a series of motions of the handheld medical instrument.

18. The ultrasound system of claim 17, wherein the motion detection device comprises at least one accelerometer.

19. The ultrasound system of claim 17, comprising at least one actuation feature integral with the housing and configured to transmit a status of the at least one actuation feature.

20. The ultrasound system of claim 19, wherein the processor is configured to perform the various operations based on detection of the motion or the series of motions of the handheld medical instrument and based on the status of the at least one actuation feature.
